(19) United States
(12) Patent Application Publication
(10) Pub. No.: US 2025/0235256 A1
(43) Pub. Date: Jul. 24, 2025

(54) GENERATOR WITH PLASMA LENGTH INDICATION

(71) Applicant: Erbe Elektromedizin GmbH, Tuebingen (DE)

(72) Inventor: Konstantin DORNHOF, Horb am Neckar (DE)

(21) Appl. No.: 19/018,120

(22) Filed: Jan. 13, 2025

(30) Foreign Application Priority Data
Jan. 23, 2024 (EP) 24153476.7

(51) Int. Cl. A61B 18/12; A61B 18/00

(52) U.S. Cl. CPC A61B 18/1206; A61B 2018/0066; A61B 2018/1286

(57) ABSTRACT

A generator having a converter module having a transformer with a the secondary winding electrically connected to an electrode of an instrument having a counter electrode. The primary winding of transformer is connected to an operating voltage (U_{b}) on one side and is connected to ground (M) via an electronic switch on the other side. The switch is capable of creating voltage impulses in the secondary winding that supply a spark or another plasma for medical treatment of a patient. A voltage detector detects the voltage (Uₚ) occurring on the primary winding. An indicator device indicates the plasma length. By detecting the voltage (Uₚ) occurring on the primary winding, the length of the plasma created can be characterized.

This application claims priority to European Patent Application No. 24153476.7, filed Jan. 23, 2024, the entirety of which is incorporated herein.

Embodiments of the invention refer to an electrosurgical generator, which is configured for carrying out an HF surgical application.

Electrosurgical generators are basically known from the prior art. For example, US 2018/0243558 A1 discloses a generator consisting of individual impulse generators, which are connected in parallel on their output side. With this generator, specifically voltage impulses can be created for electrosurgical treatment of a patient.

In addition, US 2011/0060329 A1 discloses an electrosurgical generator for creation of a treatment direct voltage. For this purpose, the generator comprises a flyback converter by means of which an output capacitor can be loaded on which the direct current builds up. Additionally, the generator can comprise multiple of such blocks in order to supply current to multiple loads.

A generator is disclosed in EP 4 147 656 A1 comprising multiple generator modules connected in series on the output side and operating according to the flyback converter principle. Each generator module comprises an externally controlled switch and a transformer, wherein the primary winding of the transformer is connected in series to the switch. The secondary windings of the different generator modules are connected in series with one another. The switch is externally controlled in a control circuit, so that it is able to build up and suddenly switch off a current in the primary winding of the transformer specifically. Due to induction, a high voltage peak is created in the secondary winding of the transformer and corresponding to the transmission ratio, a smaller voltage peak is created on the primary winding. In order to avoid a destruction of the switch due to too high primary voltage peaks, a protection capacitor is connected in parallel to the switch.

Additional prior art is formed by US 2015/0133912 A1, U.S. Pat. No. 4,878,493 and WO 2011/146498 A2.

At least one of the above-mentioned electrosurgical generators are suitable to provide an instrument with voltage which comprises an electrode for treatment of biological tissue. For treatment, an electrical spark or plasma can be maintained between the tissue and the electrode, the length of which influences the physiological effect. The electrode can be arranged in a gas stream, particularly an inert gas stream, for example, an argon stream, which is directed onto the biological tissue thereby flowing along the electrode. Such instruments are denoted as argon plasma probes or argon plasma instruments.

An example of such an argon plasma instrument is known from EP 1 684 653 B1, for example. This instrument comprises a tube or hose-shaped base body that comprises a channel for gas and plasma guidance opening out at the distal end. Inside the channel an electrode for ionization of the gas jet is arranged, so that the instrument produces a plasma jet which exits from the instrument in distal direction.

Additional instruments are known from DE 100 30 111 A1 or EP 1 293 170 A1.

During treatment of biological tissue, the view on the treatment site can be limited for the treating person. Therefore, it is sometimes difficult for the treating person to estimate the length of the created plasma. The length of the created plasma can, however, have remarkable influence on the achieved treatment result.

The object is to indicate the length of the created plasma to the treating person, also if the view on the treatment site and the created plasma is limited.

The electrosurgical generator according to an embodiment of the invention is configured for supply of HF surgical instruments, particularly instruments creating a plasma jet. For this purpose, the generator comprises at least one converter module that is connected to a direct voltage on the input side in order to be supplied therefrom with electrical power. The converter module comprises an output configured for output of a sequence of high voltage impulses. The pulse sequence frequency can thereby exceed a minimum frequency of 100 kHz, so that the impulse sequence forms a high frequency high voltage. Particularly, a non-sinusoidally formed high voltage can be created.

The converter module comprises a transformer having a primary winding and a secondary winding. While the secondary winding forms the output of the converter module, the primary winding is connected to an externally controlled electronic switch. The latter comprises a control electrode connected to a control circuit and a controlled path that connects a connection of the primary winding with ground (in controlled manner). The other connection of the primary winding is connected with the operating voltage.

Between the controlled path and the end of the primary winding connected to the controlled path, a connection point is formed to which a voltage detector is connected. This voltage detector is configured to detect the voltage occurring at this connection point. The voltage detector additionally comprises an indicator device configured to generate a signal depending on the voltage detected by the voltage detector. The signal is a signal perceptible by the treating person, for example, an optically or acoustically or also haptically perceptible signal. Combinations of such signals are possible as well.

The voltage detected by the voltage detector depends on the length of the created plasma. This plasma can form between the electrode of an instrument connected to the generator and biological tissue, which is also connected to the generator output via a neutral electrode. This applies to monopolar instruments. However, also bipolar instruments with two electrodes are possible that are connected to the generator and between which the plasma is formed.

The treating person can draw conclusions about the length of the plasma present at the instrument based on the signal provided to him/her and can orientate his/her actions thereupon. For example, he/she can in this manner avoid the production of too long plasma lengths or also the creation of too short plasma lengths up to the direct contact between the electrode of the instrument and the tissue. Embodiments of the invention supports the treating person to control the appropriate distance between the instrument or the electrode and the tissue and to maintain an appropriate distance during treatment.

The voltage detector can be a peak voltage detector that indicates the maximum voltage occurring at the connection point. Moreover, the latter can be configured as sample-and-hold circuit in which the voltage measurement carried out by the voltage detector is synchronized with the switching of the externally controlled switch. For this purpose, the control circuit controlling the switch can be connected with the voltage detector. In this manner, the voltage detector can receive control impulses from the control circuit.

If the voltage detector is a peak voltage detector, it typically comprises a storage capacitor that is connected with the connection point via a current path in order to load on the peak voltage applied there. If the voltage detector is a sample-and-hold circuit, a discharge current path can be connected in parallel to the storage capacitor. Therein a controlled discharge switch can be provided that can be controlled by the control circuit in order to discharge the storage capacitor, always shortly prior to the point in time at which the controlled switch of the converter module is blocked and thus a new peak voltage is reached.

The generator can comprise multiple converter modules of the described configuration. Preferably, these converter modules are supplied by a direct voltage with energy or power in parallel manner. On the output side, the converter modules are preferably connected in series. Each converter module can “fire” individually, that means output a high voltage impulse at its output if its controlled switch blocks for a short period. A “firing sequence” is in this case a sequence of high voltage impulses in which each converter module involved in the firing sequence has fired one time or multiple times. The firing sequence corresponds to a sequence of control signals created and supplied to the switches of the converter modules by the control circuit. The control circuit can be configured to periodically repeat the sequence of control signals any number of times for producing an HF output voltage. Accordingly, the HF output voltage is created by continuous repetition of the firing sequence.

The control circuit is preferably configured to block the control switches of the converter modules during a firing sequence individually or in groups, whereby the other switches of the other converter modules are then preferably maintained in a conducting condition. In doing so, the high voltage outputs of the converter modules, which are not firing at present, are passable for the high voltage impulses of the firing converter modules. A converter module fires if its controlled switch is blocked (preferably for a short period) and a high voltage output impulse is created in this manner.

Each of these converter modules can comprise a voltage detector according to the described configuration. However, it has shown that it can be sufficient to connect one single converter module or only some of the converter modules with the mentioned voltage detector.

Additional details of embodiments of the invention are derived from dependent claims as well as the description and the associated drawing. The drawing shows:

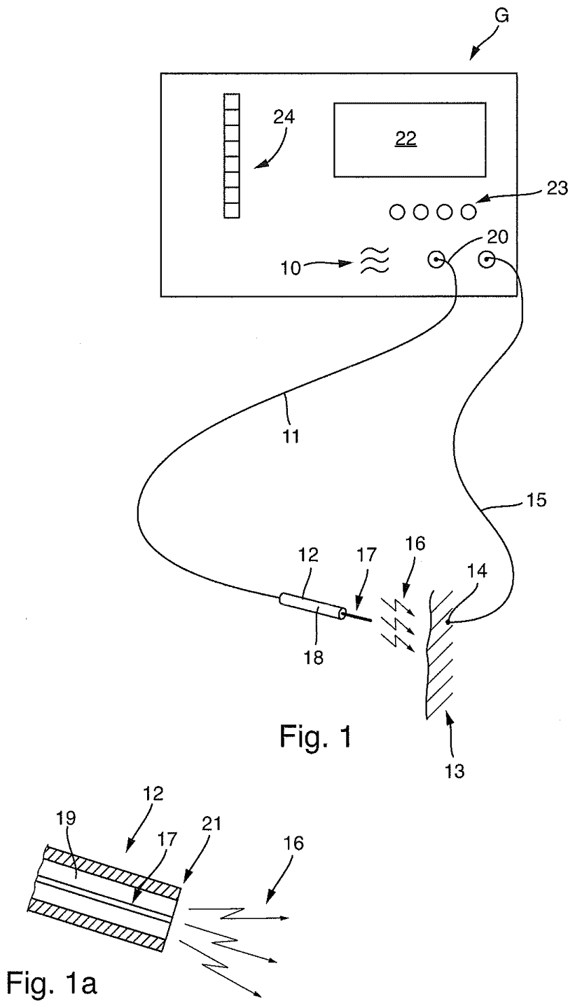

FIG. 1 illustrates the generator according to an embodiment of the invention and an instrument connected thereto in basic illustration,

FIG. 1a illustrates the distal end of an instrument during the operation in a longitudinally cut illustration,

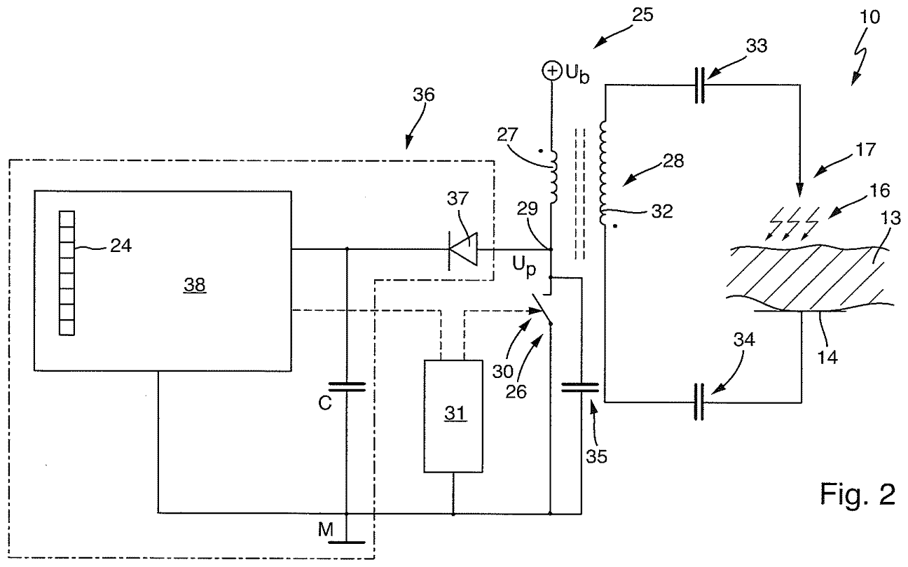

FIG. 2 illustrates a generator having one single converter module and a plasma length indication in a schematic block diagram,

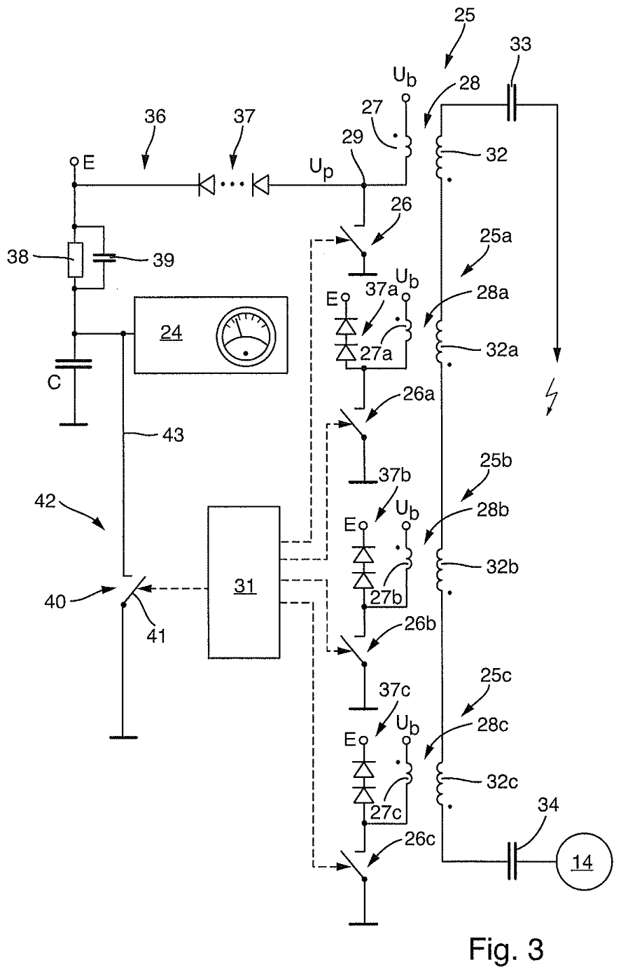

FIG. 3 illustrates a generator having multiple converter modules and a voltage detector in basic illustration,

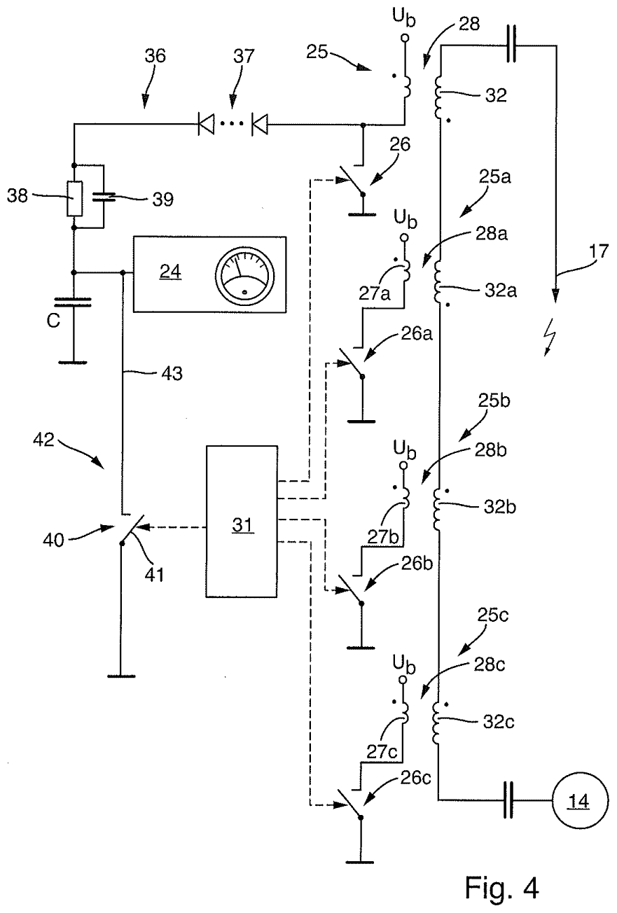

FIG. 4 illustrates a modified generator according to FIG. 3 in basic illustration,

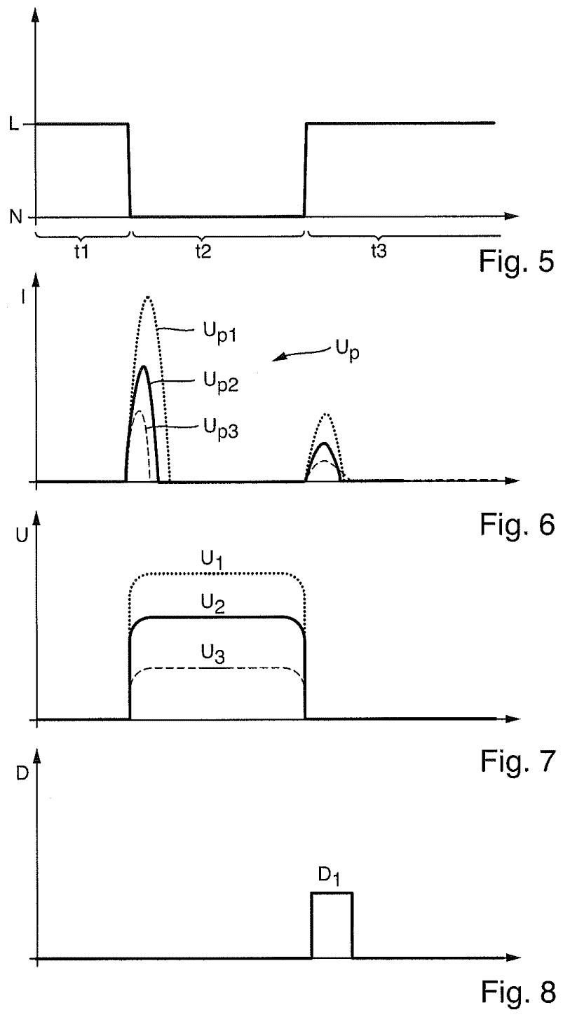

FIGS. 5 to 8 illustrate switching diagrams and voltages of the generator according to FIGS. 2 to 4, respectively,

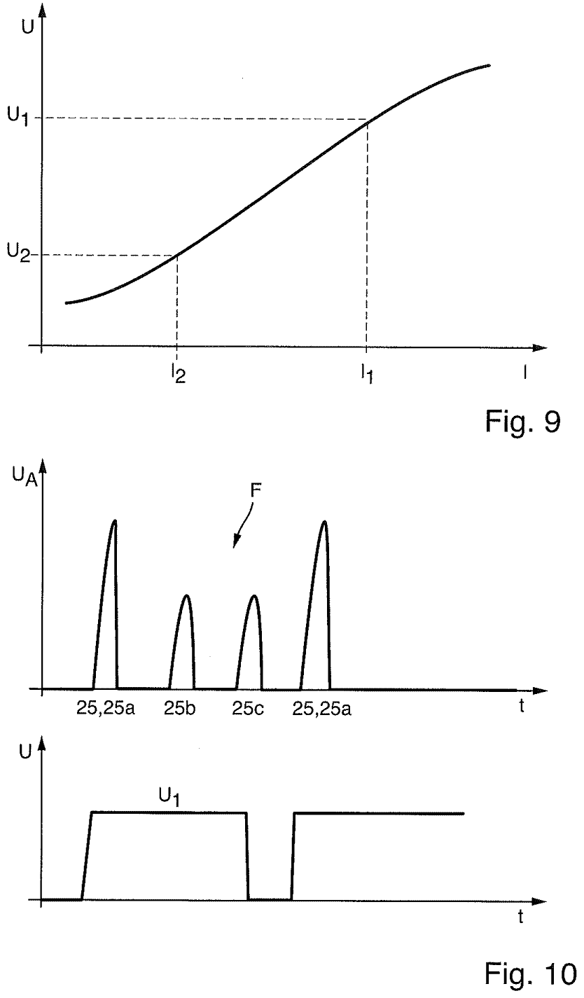

FIG. 9 illustrates a correlation between the voltage detected by the voltage detector and the plasma length,

FIG. 10 illustrates examples for firing sequences of the converter modules and the operation of the voltage detector.

In FIG. 1 an apparatus G having a generator 10 according to an embodiment of the invention is illustrated, to which an instrument 12 for treatment of biological tissue 13 is connected via a line 11. The biological tissue 13 is connected to the generator 10 via a neutral electrode 14 and a line 15. FIG. 1, therefore, illustrates a monopolar instrument in which the current flows from instrument 12 toward the tissue 13. Basically, however, embodiments of the invention can also be applied to bipolar or multiple polar instruments, which influence biological tissue by a plasma 16. In FIG. 1 plasma 16 is illustrated by jagged arrows.

The instrument 12 comprises an electrode 17 for plasma creation, which is electrically connected with generator 10 via line 11. In FIG. 1 instrument 12 is illustrated as handle having an electrode 17 projecting therefrom. Such instruments are suitable for the open surgical use. Thereby, electrode 17 can extend away from handle 18 and can be configured in the type of a needle or scalpel. However, it is also possible to arrange electrode 17 inside a channel 19, which extends through the instrument 12 and is connected at the proximal end 20 to the supplying apparatus G that, in this case, comprises a respective gas supply in addition to generator 10.

At the distal end 21 (FIG. 1a) gas, which is supplied by apparatus G into the channel 19, can flow out. It can be ionized by electrode 17 so that an exiting plasma stream 16 is formed that is again illustrated by jagged arrows. Thereby, the instrument 12 can be an instrument configured for the open surgical use, as illustrated in FIG. 1, as well as a laparoscopic instrument, as well as a probe that is guided through the working channel of an endoscope toward the operation site, for example. However, it is common to all these uses that the treating person does not always have unlimited free view on the plasma stream 16 and its length.

As illustrated in FIG. 1, apparatus G comprises a display device 22 as well as operation elements 23, for example push buttons, knobs or the like. In addition, apparatus G comprises an indicator device 24 that serves for indication of the length of the plasma stream 16. The indicator device 24 is illustrated as optical indicator device in FIG. 1 that shows a light bar which symbolizes the length of the plasma stream 16. In addition, the light bar can have at least at one of its ends colored fields by which it signalizes a too long and/or too short plasma stream 16.

The indicator device 24 can be configured as optical indicator device and can also be integrated in the display device 22 if required. In addition, it is possible to additionally or alternatively provide the indicator device 22 with an acoustic indicator. Additionally or alternatively, the indicator device 24 can be configured to generate haptically perceptible signals, for example, noticeable vibrations in the handle 18 or at another position.

FIG. 2 illustrates a part of the electrical circuit of generator 10 in simplified illustration. The already introduced reference signs are used in the following with the same meaning.

The electrosurgical generator 10 comprises a converter module 25, part of which is an electrical circuit configured in the type of a flyback converter. The latter is formed by an externally controlled electronic switch 26, for example in the form of a field effect transistor or a bipolar transistor or a bipolar transistor having an insulated gate or another electronic switch, which is connected in series with a primary winding 27 of a transformer 28. Thereby a first end of the primary winding 27 is connected with a positive operating voltage U_{b}, while the other end of the primary winding 27 is connected with a connection point 29. At this connection point 29, the externally controlled electronic switch is connected with one end (drain or collector) while the other end of its controlled path (source or emitter) is connected with ground potential M.

The externally controlled switch 26 additionally comprises a control electrode 30 connected with a control circuit 31, in order to deblock or block the controlled path of the electronic switch 26 in a controlled manner.

The transformer 28 comprises additionally a secondary winding 32, one end of which is connected with electrode 17 of instrument 12, where appropriate via a coupling capacitor 33. The other end of the secondary winding is connected with the neutral electrode 14, where appropriate via an additional coupling capacitor 34, whereby the neutral electrode 14 is to be attached on the patient and thus on the biological tissue 13. The secondary winding 32 or, if present, the electrodes of the coupling capacitors 33, 34, facing away from the secondary winding, form the output of the converter module 25 and thus the generator 10.

A protection capacitor 35 or also another protection circuit can be connected in parallel to the electronic switch 26. The protection capacitor 35 is connected with one connection to the connection point 29 and with its other connection to ground. It serves to limit the voltage on switch 26.

To the connection point 29, a voltage detector is connected, which is configured to detect the voltage Uₚ occurring at the connection point 29 and thus on the switch 26 (as on the protection capacitor 35 as well). The indicator device 24 is part of the voltage detector 36 and serves to create a signal depending on the detected voltage Uₚ (FIG. 6) and characterizes its amount. For example, indicator device 24 can be arranged on the apparatus G or the generator 10 or also on the instrument 12 or at a separate location.

Preferably, voltage detector 36 is configured as integrating detector or also as peak voltage detector. It comprises a storage capacitor C that is connected to the connection point 29 via a loading circuit 37. In the simplest case, the loading circuit 37 is a diode that is poled in flow direction with regard to the voltage Uₚ occurring at the connection point 29. Preferably, a diode is used having remarkably low capacitance. For reducing parasitic capacitance of the load circuit 37, multiple diodes can be connected in series with each other.

Parallel to the storage capacitor C, an evaluation circuit 38 is connected that indicates the voltage of capacitor C by the indicator device 24 on one hand and is configured on the other hand to discharge the storage capacitor C periodically.

The generator described so far operates as follows: For treatment of tissue 13, electrode 17 is brought in proximity to the tissue 13. Now, control circuit 31 opens and closes switch 26 in quick sequence and, in this manner, makes converter module 25 to fire. If switch 26 is conducting, a current flows from the (for example positive) operating voltage U_{b} through primary winding 27 from the beginning of the winding, characterized by a dot, to the end of the winding and, thus, via the connection point 29 and the conducting switch 26 to ground potential M. Every time the control circuit 31 supplies a blocking impulse to the control electrode 30, switch 26 blocks so that a further current flow to ground becomes impossible. The current flow is now commutated to the secondary winding 32, where the current flows continuously from the beginning of the winding, characterized by the dot, to the end of the winding (and, thus, to the coupling capacitor 33) and via the letter to the electrode 17.

Due to the spark discharge to the tissue 13 resulting from the created high voltage impulses, plasma 16 is created that can burn either in air, in vapor, or also in specifically supplied gas, for example argon. The voltage that builds up between electrode 17 and the tissue 13 or the neutral electrode 14, is thereby a measure for the length of plasma 16. The voltage applied between electrode 17 and neutral electrode 14 is also present at secondary winding 32. Corresponding to the transmission ratio of transformer 28, the respective voltage Uₚ is also present on the primary winding 27 and in form of a respective voltage impulse, thus, at the connection point 29. For explanation purposes, reference is made to the diagrams according to FIGS. 5 and 6:

In FIG. 5 switch 26 is conductive during time period t1. The conductive condition is characterized on the ordinate with capital letter L. At the end of the time phase t1 switch 26 is blocked. A blocking phase starts characterized by time phase t2 in FIG. 5. The non-conductive condition is characterized on the ordinate by letter N. At the beginning of blocking phase t2, the described voltage impulse Uₚ is created at primary winding 27 and respectively increased also at secondary winding 32. This voltage impulse Uₚ is illustrated in FIG. 6 in three different amounts Uₚ₁, Uₚ₂, Uₚ₃, which corresponds to three different plasma lengths of plasma 16. It thereby shows that the voltage impulse Uₚ is higher the longer the plasma 16.

FIG. 7 illustrates for this purpose voltage U on storage capacitor C. During voltage impulse Uₚ, current flows on the storage capacitor C via loading circuit 37 and loads storage capacitor C up to a respective voltage U1, U2 or U3. The higher the voltage impulse Uₚ is, the higher the voltage U on storage capacitor C.

If required, storage capacitor C can be discharged from time to time. For example, it can be discharged during or after switching on switch 26 again, occurring during the conductive phase t3, according to FIG. 5. FIG. 8 illustrates such a discharge impulse D1.

The indicator device 24 is configured to create a signal corresponding to the voltage U1, U2 or U3, for example in that a light bar represented by the indicator device 24 changes its length according to the voltage U on storage capacitor C in steps or continuously.

FIG. 3 illustrates a further developed type of generator according to FIG. 2, whereby the above description applies while maintaining the already introduced reference signs and under additional consideration of the following explanation:

The generator according to FIG. 3 comprises multiple converter modules 25, 25a, 25b, 25c, whereby the converter modules 25a, 25b, 25c are preferably identical in construction to the converter module 25 and the above description applies under addition of a respective letter index.

The loading circuit 37 is at least connected to the connection point 29 and leads via a series connection of diodes poled in flow direction to circuit point E. From this circuit point E, the current path preferably leads to the storage capacitor C via a resistor 38 and a capacitor 39 connected in parallel. The indicator device 24 is connected to this storage capacitor C.

For occasional discharge, regular discharge or discharge as required of storage capacitor C, a discharge circuit 42 having a discharge current path 43 is provided in which a discharge switch 40 is arranged. The discharge switch 40 comprises a controlled path connected in parallel to the storage capacitor C. Its control electrode 41 is connected to the control circuit 31 that does not only control all switches 26, 26a, 26b, 26c, but also the discharge switch 40. In this case, impulse D1, illustrated in FIG. 8, symbolizes the time phase during which the discharge switch 40 is deblocked, that means is conductive. Thereby, it allows the charge of the storage capacitor C to drain, whereby the voltage U1, U2 or U3 collapses, that means drops down close to zero.

In many cases it is sufficient if the loading circuit 37 only connects the connection point 29 with circuit point E. However, it can be useful to also connect one or additional of the converter modules 25a, 25b, 25c, having respective loading circuits 27a, 27b, 27c with circuit point E.

The control circuit 31 can be configured to activate and deactivate the converter modules 25, 25a, 25b, 25c in a coordinated manner, that means to open and close their respective switches 26, 26a, 26b, 26c. For this purpose, the individual switches 26, 26a, 26b, 26c can be individually transferred from their conductive condition during a short period in the blocking phase t2 according to FIG. 5, in order to produce a voltage impulse on its respective secondary winding 32, 32a, 32b or 32c, that means to fire. Preferably the switches of the respective converter modules 25, 25a, 25b, 25c that do not fire are conductive. In doing so, the high voltage impulse output from the respectively firing converter module 25, 25a, 25b, 25c can pass the low ohmic secondary windings of the non-firing converter modules, so that the high voltage impulse can flow through the secondary windings 32, 32a, 32b, 32c connected in series with each other.

In relation thereto FIG. 10 illustrates a firing sequence F based on voltage U17 at the electrode 17. The converter modules 25 and 25a are firing concurrently, subsequently the converter modules 25b, 25c. The voltage detector 36 connected to the converter module 25 detects the voltage Uₚ, whereby the capacitor C is loaded accordingly. The charge is maintained until it is unloaded again, in that the control circuit 31 activates discharge switch 40. The control circuit 31 can do this at the end of the firing sequence F if only voltage Uₚ at one of the converter modules is monitored. If multiple or all of the converter modules 25, 25a, 25b, 25c are connected to the voltage detector 36, the capacitor can also be discharged prior to the firing of the next converter module, in each case.

Typically, control circuit 31 causes the converter modules 25, 25a, 25b, 25c to fire according to a preset scheme individually or in groups. Thereby, high voltage impulses of equal or different amount can be created, as illustrated in FIG. 10. If the discharge of storage capacitor C only occurs after termination of a firing sequence, a voltage is built up on storage capacitor C that is characterized by the highest of the created high voltage impulses present in the firing sequence F. This voltage characterizes the light arc length or plasma length indicated by indicator device 24.

FIG. 9 illustrates the correlation between the voltage U_{c} on storage capacitor C and the light arc length l. With different arc lengths l₁, l₂, different voltages U1, U2 result, whereby the relation is largely linear, at least in a limited, however relatively wide range. Independent from the linearity, the voltage U_{c} is a measure for the light arc length l.

Another modified embodiment of the generator according to the invention is illustrated in FIG. 4. The already introduced reference signs also apply for this embodiment, because the description related thereto applies accordingly. The difference between the embodiment according to FIG. 4 and the embodiment according to FIG. 3 is (solely) the polarity of the secondary windings 32, 32a, 32b, 32c. While in the embodiment according to FIG. 3 all of the secondary windings 32, 32a, 32b, 32c are identically poled, in that the beginnings of the windings, characterized by a dot, are orientated toward neutral electrode 14, different polarities are present in the embodiment according to FIG. 4. The secondary windings 32, 32a are facing neutral electrode 14 with their beginnings of the windings, while the secondary windings 32b, 32c are facing electrode 17 with their beginnings of the windings. However, it is to be noted that also other groupings and polarities can be provided.

In the embodiment according to FIG. 4, converter modules 25, 25a create positive voltage impulses at electrode 17 if they are firing, while the converter modules 25b, 25c create negative voltage impulses at the electrode 17. (this applies in case of positive operating voltage U_{b}, in case of negative operating voltage U_{b}, the circumstances are vice versa.)

Due to the combination of converter modules 25, 25a, 25b, 25c with different polarity symmetric as well as asymmetric HF output voltage sequences can be created. Also, in case of symmetric HF impulse sequences that contain positive as well as negative voltage peaks, the indication of the plasma length succeeds, as described above, by detection of voltage Uₚ at the appropriate connection points 29 and/or 29a, 29b, 29c.

The generator 10 according to an embodiment of the invention comprises at least one converter module having a transformer 28, the secondary winding 32 of which is electrically connected with the electrode 17 of an instrument 12 and with a counter electrode 14. The primary winding 27 of transformer 28 is connected to an operating voltage U_{b} on one side and is connected to ground M via an electronic switch 26 on the other side. The switch 26 blocks periodically, whereby voltage impulses are created in the secondary winding 32 that supply a spark or another plasma for medical treatment of a patient. A voltage detector 36 detecting the voltage Uₚ occurring on the primary winding 27 and indicates it by an indicator device 24 serves for indication of the plasma length. An embodiment of the invention is based on the concept that the voltage Uₚ occurring on the primary winding 27, particularly the peak voltage, characterizes the length of the created plasma 16. The inventor has found out that other influencing parameters than the plasma length are of subordinate importance on the voltage Uₚ measured at the primary winding 27.

1. An electrosurgical generator, comprising:
a converter module having an externally controlled switch connected to a control circuit and a transformer, the transformer having a primary winding connected in series to the switch and a secondary winding connected to an electrode and a counter electrode, whereby the electrode and the counter electrode are configured to accommodate a patient between;
a voltage detector connected to the switch configured to detect the occurring voltage (Uₚ); and
an indicator device configured to generate a signal depending on the detected voltage (Uₚ).

2. The electrosurgical generator according to claim 1, wherein the switch is connected to a reference potential (M) with one end and a connection point with another end, whereby the connection point is connected with an end of the primary winding and the voltage detector is connected to the connection point.

3. The electrosurgical generator according to claim 1, wherein the voltage detector is configured to measure peak voltage.

4. The electrosurgical generator according to claim 1, wherein the voltage detector is a sample-and-hold-circuit.

5. The electrosurgical generator according to claim 1, wherein the voltage detector comprises a storage capacitor (C) that is connected with the switch via a loading circuit.

6. The electrosurgical generator according to claim 5, wherein the loading circuit has at least one diode.

7. The electrosurgical generator according to claim 1, wherein a discharge circuit is assigned to the storage capacitor (C), wherein the discharge circuit comprises a controllable discharge current path connected in parallel to the storage capacitor (C).

8. The electrosurgical generator according to claim 7, wherein the discharge circuit is connected to the control circuit and configured such that its discharge current path can be switched between a non-conductive and a conductive condition by the control circuit.

9. The electrosurgical generator according to claim 8, wherein the control circuit is configured to alternatingly open and close the externally controlled switch, wherein it is additionally configured to also block the discharge current path when the switch is blocked.

10. The electrosurgical generator according to claim 9, wherein the control circuit is configured to temporarily open the discharge current path prior to the blocking of the switch.

11. The electrosurgical generator according to claim 10, further comprising a second converter module having a secondary winding connected in series to the secondary winding of the first converter module.

12. The electrosurgical generator according to claim 11, the converter modules are of identical construction.

13. The electrosurgical generator according to claim 11, wherein the secondary windings are connected in series with equal orientation.

14. The electrosurgical generator according to claim 11, wherein the secondary windings connected in series with opposite orientation.

15. The electrosurgical generator according to claim 11, wherein the voltage detector is connected to only one of the converter modules.
